(19) United States
(12) Patent Application Publication
(10) Pub. No.: US 2019/0083322 A1
(43) Pub. Date: Mar. 21, 2019

(54) NEGATIVE PRESSURE WOUND DRESSING

(71) Applicant: BenQ Materials Corporation, Taoyuan (TW)

(72) Inventors: Mao-Sung Huang, Taoyuan City (TW); Huai-Wei Hsieh, Taoyuan City (TW)

(21) Appl. No.: 15/925,690

(22) Filed: Mar. 19, 2018

(30) Foreign Application Priority Data
Sep. 21, 2017 (TW) 106214057

(51) Int. Cl. A61F 13/00; A61F 13/02; A61M 1/00; A61L 15/42; A61L 15/22; A61L 15/60

(52) U.S. Cl. CPC A61F 13/00055; A61F 13/0206; A61F 13/00059; A61F 13/0209; A61F 13/0213; A61F 2013/00131; A61M 1/0088; A61L 15/425; A61L 15/225; A61L 15/60; A61F 13/0216

(57) ABSTRACT

A negative pressure wound dressing is provided. The negative pressure wound dressing sequentially includes a barrier layer, an indicating layer, a liquid impermeable, a liquid impermeable, gas permeable layer, an absorbent layer and a wound contacting layer, wherein, the barrier layer comprises a connecting hole for fluidly connecting a negative pressure source, and the indicating layer comprises at least one visual indicating element on the surface adjacent to the barrier layer. By using the negative pressure wound dressing of the present invention, the user can be reminded to replace the dressing timely by the visual indicating element which can detect the wound exudates absorption in the absorbing layer to effectively improve the wound healing.

### RELATED APPLICATIONS

This application claims priority to Taiwanese Application Serial Number 106214057, filed on Sep. 21, 2017, which is incorporated herein by reference.

### BACKGROUND OF THE INVENTION

### Field of Invention

The invention relates to a negative pressure wound dressing, and more particularly relates to a negative pressure wound dressing with a visual indicating element.

### Description of Related Art

Wound healing and repair is a complex process where the skin or other body tissue heals and repairs itself after injury. Firstly, the injury damaged to the body can be minimized by delivering proteins and other clotting substances to the wound by the blood. Blood clots are provided to stop bleeding, at the same time, and bacteria and other pathogens or debris are phagocytosed by white blood cells. And then, the body begins to heal and repair itself during a healing phase called proliferation, wherein the granulation tissue is formed on the epidermis of the wound to provide a basic structure for cells to migrate from the periphery of the wound into the interior of the wound to heal the wound. Finally, scars will be formed with time.

The blood and tissue fluid discharged from the wound are usually absorbed by the conventional wound dressing to maintain dryness of the wound by physical adsorption method. However, the conventional wound dressing cannot control the environment humidity around the wound, and excessively dry environment will affect the wound healing.

A method for controlling the environment humidity to enhance wound healing in proliferation phase called negative pressure wound therapy (NPWT) was disclosed. Negative pressure wound therapy is a therapeutic technique using a simple negative pressure source, such as vacuum pump, combined with sealed drape, soft suction head and biocompatible porous material to form a negative pressure environment for extracting pus and infectious materials discharged from the wound, attracting healthy tissue fluid to maintain wet therapeutic environment and increasing capillary circulation around the wound to enhance the wound healing. The negative pressure wound therapy uses natural healing processes of the body to enhance forming granulation tissue on the wound and migration of healthy tissue by effectively increasing blood flow to the wound. The negative pressure wound therapy can inhibit the growth of bacteria by removing exudates discharged from the wound.

The conventional negative pressure wound therapies can be classified into wound exudates canister system and absorbent dressing system. US patent publication NO. US 2011/0213287 discloses a negative pressure wound system comprising a negative pressure source, a dressing and a canister between the negative pressure source and the dressing for collecting the wound exudates. However, the negative pressure wound system comprising canister needs more space for disposing the extra canister, connecting tube and support, which is not convenient to use and the movements of patients will be affected. CN patent publication NO. CN10246119 discloses a multilayer dressing for negative pressure wound system which comprises an absorbent layer and a water blocking layer, the wound exudates sucked by the negative wound dressing can be contained in the absorbent layer and will not be sucked into the negative pressure source due to the water blocking layer. However, when excess wound exudates are absorbed into the space of the absorbent layer, the suction efficiency of the negative pressure source will be affected.

In view of the foregoing, there is a need for a negative pressure wound dressing containing a visual indicating element which is helpful to remind the user to replace the dressing timely. Therefore, the problem of obstructing the negative pressure delivery and the detriment of the expected healing and repair effect due to the excess wound exudates in the absorbent layer can be avoided.

### SUMMARY OF THE INVENTION

The present invention is directed to a novel negative pressure wound dressing, the user can be reminded to replace the dressing timely by the visual indicating element for improving the wound healing efficiency.

According to an aspect of the present invention, a negative pressure wound dressing is provided. In an embodiment of the present invention, the negative pressure wound dressing includes a barrier layer comprising a connecting aperture for fluidly communicating to a negative pressure source; an indicating layer is stacked on the barrier layer, wherein the indicating layer comprises at least one visual indicating element on the surface adjacent to the barrier layer; a liquid impermeable, gas permeable layer stacked on the indicating layer; an absorbent layer stacked on the liquid impermeable, gas permeable layer; and a wound contacting layer stacked on the absorbent layer.

In an embodiment of the present invention, the visual indicating element is a humidity sensing element.

In an embodiment of the present invention, the humidity sensing element is a pattern of water based ink.

In an embodiment of the present invention, the humidity sensing element is a pattern of humidity sensing agent, an aqueous-triggered color-appearing ink, a hydrochromic ink or soaking discoloration ink.

In an embodiment of the present invention, the liquid impermeable, gas permeable layer comprising a plurality of micropores with a diameter in the range of 0.3 μm to 0.6 μm.

In an embodiment of the present invention, the liquid impermeable, gas permeable layer can be a polymer film of polyethylene, polypropylene, polyvinyl alcohol, polyvinyl acetate, polytetrafluoroethylene, polyvinylidene fluoride, polyvinyl pyrrolidone, polyurethanes, polyamides, polyesters, polyacrylates, polymethacrylate, polyacrylamide or combinations thereof.

In an embodiment of the present invention, the absorbent layer is able to hold at least 30 milliliters of fluid.

In an embodiment of the present invention, the material of the absorbent layer can be the form of fabric, sponge, foam, and made from such as super absorbent polymers, hydrogel materials or gel materials.

In an embodiment of the present invention, the wound contacting layer comprises a plurality of vertical through holes for the exudates to flow.

In an embodiment of the present invention, the materials for the wound contacting layer can be polyurethane, polyolefin, vinyl acetate, silicone, fluorosilicone, fluoroelastomers, styrene, butadiene, thermoplastic elastomers or combinations thereof.

In an embodiment of the present invention, the barrier layer is an impermeable elastic film.

In an embodiment of the present invention, the materials for the barrier layer can be natural rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane, ethylene vinyl acetate, co-polyester, silicones or combinations thereof.

In another embodiment of the present invention, the negative pressure wound dressing further comprises a first pressure distributing layer stacked between the indicating layer and the liquid impermeable, gas permeable layer.

In an embodiment of the present invention, the material of the first pressure distributing layer can be natural or synthetic reticulated foam materials, non-woven fabrics or woven fabrics.

In further another embodiment of the present invention, the negative pressure wound dressing further comprises a second pressure distributing layer stacked between the liquid impermeable, gas permeable layer and the absorbent layer.

In an embodiment of the present invention, the material of the second pressure distributing layer can be natural or synthetic reticulated foam materials, non-woven fabrics or woven fabrics.

In still further another embodiment of the present invention, the negative pressure wound dressing comprises a third pressure distributing layer stacked between the absorbent layer and the wound contacting layer.

In an embodiment of the present invention, the material of the third pressure distributing layer can be natural or synthetic reticulated foam materials, non-woven fabrics or woven fabrics.

The forgoing presents a simplified summary of the disclosure in order to provide a basic understanding of the present invention. This summary is not an extensive overview of the disclosure and it does not identify key/critical elements of the present invention or delineate the scope of the present invention. Its sole purpose is to present concepts disclosed herein in a simplified form as a prelude to the more detailed description that is presented later. Many of the attendant features will be more readily appreciated as the same becomes better understood by reference to the following detailed description considered in connection with the accompanying drawings. It is noteworthy that the drawings shown in the figures are for illustrative purposes only and not to scale.

### BRIEF DESCRIPTION OF THE DRAWINGS

The invention can be more fully understood by reading the following detailed description of the embodiment, with reference made to the accompanying drawings as follows:

FIG. 1 is a cross-sectional view of the negative pressure wound dressing according to an embodiment of the present invention.

FIG. 2 is a top view of the indicating layer of the negative pressure wound dressing according to an embodiment of the present invention.

FIG. 3 is a cross-sectional view of the negative pressure wound dressing according to another embodiment of the present invention.

FIG. 4 is a cross-sectional view of the negative pressure wound dressing according to further another embodiment of the present invention.

### DETAILED DESCRIPTION

Referring now to the drawings to illustrate the embodiments of the present negative pressure wound dressing. In the following embodiments of the present disclosure, the like elements refer to like symbols in the figures. The following description will introduce the embodiment of the above-described negative pressure wound dressing. For being better understood the embodiments of the present disclosure, a detailed description thereof is provided. However, well-known functions or constructions may not be described in detail for brevity and/or clarity. The drawings are not necessarily to scale, emphasis instead being placed upon illustrating the principles of the disclosure rather than to limit and restrict of the scope of the present disclosure defined in the appended claim.

The advantages and features of the present disclosure will become better understood with reference to the following more detailed description and claims taken in conjunction with the accompanying drawings. It should be understood that the exemplary embodiments of the present disclosure described below may be modified in many different ways without departing from the inventive principles disclosed herein, and the scope of the present disclosure is therefore not limited to these particular embodiments. Rather, these embodiments are provided so that this disclosure will be thorough and complete, and will fully convey the concept of the disclosure to those skilled in the art by way of example and not of limitation. The present disclosure is only defined by the appended claims.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein.

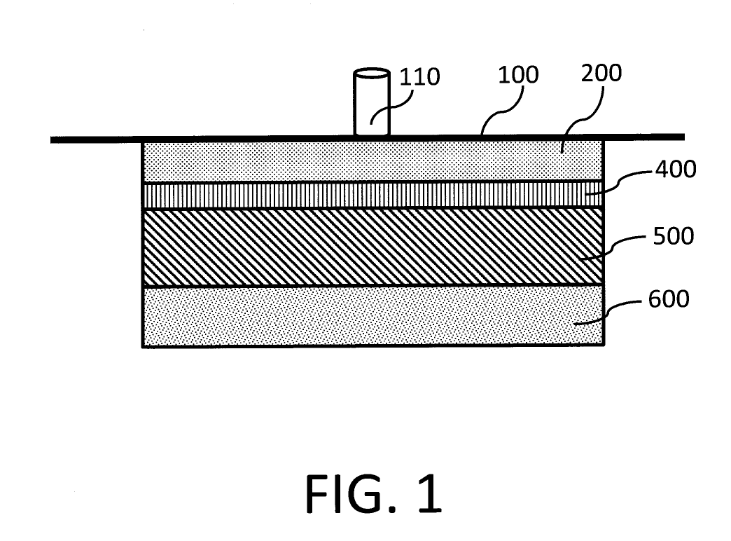

FIG. 1 is a cross-sectional view of a negative pressure wound dressing according to an embodiment of the present invention. As illustrated in FIG. 1, the negative pressure wound dressing sequentially comprises a barrier layer 100, an indicating layer 200, a liquid impermeable, gas permeable layer 400, an absorbent layer 500 and a wound contacting layer 600.

As used herein, “negative pressure” refers to a pressure less than the ambient pressure at a tissue site that is being subjected to treatment. In most cases, this negative pressure will be less than the atmospheric pressure at which the patient is located. Alternatively, the negative pressure may be less than a hydrostatic pressure at the tissue site. The negative pressure may be constant or varied and which may be delivered continuously or intermittently.

The barrier layer 100 is disposed to cover a wound. In an embodiment of the present invention, the periphery of the barrier layer 100 facing the wound has an adhesive (not shown) used to seal the wound area to provide a fluid sealed environment. In another embodiment of the present invention, the barrier layer 100 is covered on the peripheral epidermis of the wound to form a fluid sealed environment by a tape. Therefore, the barrier layer 100 can act as a microbial barrier to prevent contaminants from entering into the wound, and fix the absorbent layer 500 and the wound contacting layer 600 above the wound. The barrier layer 100 comprises a connecting aperture 110 for fluidly communicating to a negative pressure source, such as a vacuum pump or another suction source.

Suitable materials for the barrier layer 100 can be a moisture permeable membrane to promote the exchange of oxygen and moisture between the atmosphere and the wound. The barrier layer 100 can also be an impermeable elastic film or rigid member. To consider the convenience of use and fitting for the wound, in an embodiment of the present invention, the barrier layer 100 is an impermeable elastic film. In an embodiment of the present invention, the materials for the barrier layer 100 can be, such as natural rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane, ethylene vinyl acetate, co-polyester or silicones.

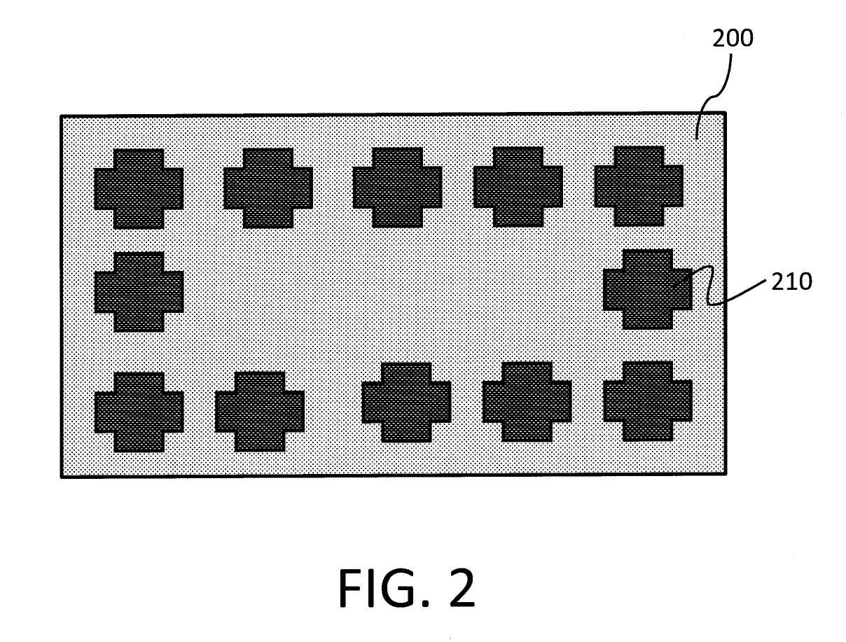

The indicating layer 200 is stacked on the barrier layer 100, wherein the indicating layer 200 comprises at least one visual indicating element 210 on the surface adjacent to the barrier layer 100, as shown in FIG. 2. In an embodiment of the present invention, the indicating layer 200 can be a gas permeable polymer film, such as but not limited to gas permeable polypropylene film for improving the negative pressure delivery. The visual indicating element 210 can be disposed by the demand of different designs. The shape, pattern, size and quantity are not limited to the figures. In an embodiment of the present invention, the visual indicating element 210 can be a humidity sensing element which will be colorless when be dissolved in water. For example, the humidity sensing element is a pattern of water based ink consisting of waterborne resin, pigment and solvent but not limited thereto. In another embodiment of the present invention, the humidity sensing element can be a pattern of humidity sensing agent, aqueous-triggered color-appearing ink, hydrochromic ink or soaking discoloration ink. Suitable humidity sensing agents can be a cobalt chloride agent which appears different colors in an aqueous environment and in an anhydrous environment. Suitable aqueous-triggered color-appearing inks can be an ink comprising water-soluble chromogenic dyes which will be transparent in an anhydrous environment and will appear specific color in an aqueous environment. Suitable hydrochromic inks can be, such as an ink comprising a color-changing agent which will appear different colors in an aqueous environment and in an anhydrous environment. Suitable soaking discoloration inks or decolorizable aqueous inks comprise dyes of water soluble oxidant or reductant which is capable of changing color in response to wetness and being colorless in an aqueous environment. When the moisture accumulated in the indicating layer 200, the visual indicating element 210 will disappear, change color, deform or fade. Therefore the user can replace the wound dressing timely by the visual indicating element 210 for detecting the humidity.

The liquid impermeable, gas permeable layer 400 is stacked on the indicating layer 200. The liquid impermeable, gas permeable layer 400 can prevent liquid from delivering but allow a gas (for example water vapor) to leave. Suitable materials for the liquid impermeable, gas permeable layer 400 can be a material which can prevent fluid from entering and leaving through the material and allow gas to leave or flow through the material. In an embodiment of the present invention, the liquid impermeable, gas permeable layer 400 comprises a plurality of micropores with a diameter in the range of 0.3 μm to 0.6 μm. The liquid impermeable, gas permeable layer 400 can be but not limited to a high water vapor transfer membrane or other structures made from hydrophilic polymers. In an embodiment of the present invention, the material of the liquid impermeable, gas permeable layer 400 can be a polymer film of polyvinyl alcohol, polyvinyl acetate, polyvinyl pyrrolidone, polyurethanes, polyamides, polyesters, polyacrylates, polymethyl methacrylate or poly acrylamide. In an embodiment of the present invention, the liquid impermeable, gas permeable layer 400 can be further treated in order to obtain hydrophobic surface characteristics for promoting gas to flow upwards and prevent fluid, particle or pathogen from entering through the liquid impermeable, gas permeable layer 400.

The conventional negative pressure wound dressing must comprises a liquid-collection canister for storing a fluid like wound exudates or tissue fluid sucked by the negative pressure. However, disposing a liquid-collection canister needs a larger space and may affect the movement of user. Accordingly, an absorbent layer 500 is stacked on the liquid impermeable, gas permeable layer 400 of this present negative pressure wound dressing. Therefore, the wound exudates or tissue fluid can be stored in the absorbent layer 500, and the liquid-collection canister can be replaced thereby. Moreover, due to the wound exudates or tissue fluid is stored in the absorbent layer 500, the wound exudates or tissue fluid can stay away from the skin of patient and a problem of wound impregnating can be avoided. The liquid-collection canister can be removed from the negative pressure wound dressing and replaced with an absorbent layer 500 which is capable of holding at least 30 milliliters of fluid to increase the convenience during surgery In an embodiment of the present invention, the material of the absorbent layer 500 can be the form of fabric, sponge or foam, made from the materials commonly used in the medical care field, such as super absorbent polymers, hydrogel materials or gel materials but not limited thereto.

The wound contacting layer 600 is stacked on the absorbent layer 500. The wound contacting layer 600 directly contacts with the wound. The wound contacting layer 600 can be but not limited to an anti-sticking polyurethane layer, a polyethylene layer, a silicone layer or other flexible layers. The wound contacting layer 600 includes a plurality of flow channels or pathways for delivering liquid and gas. The wound contacting layer 600 is provided to assist in preventing the tissue from growing into the wound dressing, applying negative pressure to the tissue, delivering fluids to the tissue, or removing fluids from the tissue site. Example of materials suitable for the wound contacting layer 600 include but not limited to polyurethanes, polyolefins, vinyl acetate, polysilicon, silicone, fluorosilicone, fluoroelastomers, styrene, butadiene or thermoplastic elastomers.

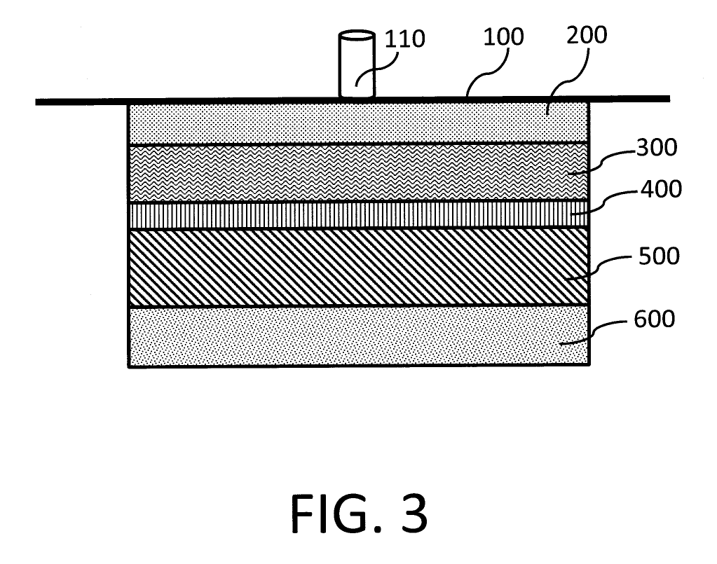

Optionally, in another embodiment of the present invention, the negative pressure wound dressing can further comprises a first pressure distributing layer 300 stacked between the indicating layer 200 and the liquid impermeable, gas permeable layer 400, as shown in FIG. 3. The first pressure distributing layer 300 is provided to assist in spreading the negative pressure generated by the negative pressure source so that the whole wound site bears an equalized negative pressure, and the water vapor drawn to the indicating layer 200 can evenly delivered, and thus, the visual indicating element 210 of the indicator layer 200 can change evenly. In an embodiment of the present invention, the materials for the first pressure distributing layer 300 can be but not limited to natural or synthetic reticulated foam materials, non-woven fabrics or woven fabrics.

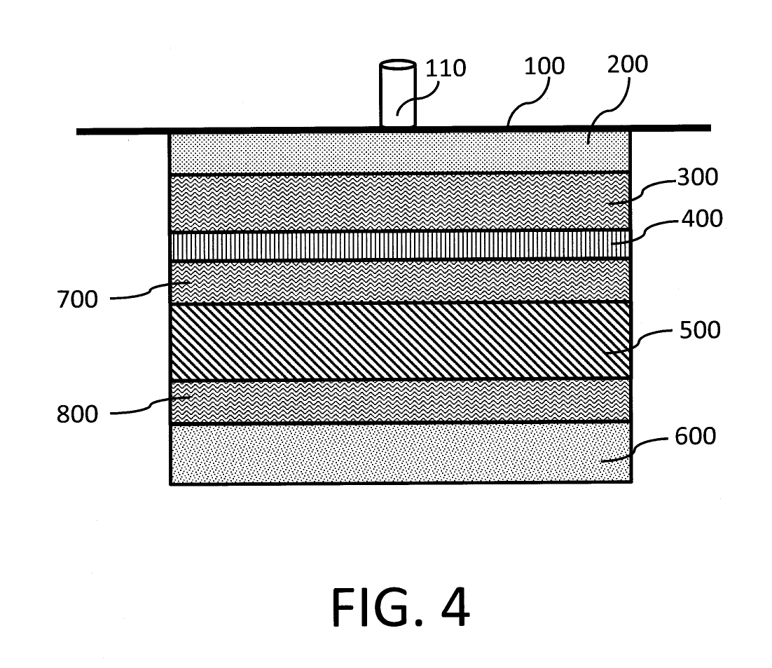

In further another embodiment of the present invention, the negative pressure wound dressing can further comprises a second pressure distributing layer 700 and a third pressure distributing layer 800 as shown in FIG. 4. The second pressure distributing layer 700 is stacked between the liquid impermeable, gas permeable layer 400 and the absorbent layer 500. The third pressure distributing layer 800 is stacked between the absorbent layer 500 and the wound contacting layer 600. The second pressure distributing layer 700 and the third pressure distributing layer 800 are provided to assist in spreading and distributing the negative pressure generated by the negative pressure source so that the whole wound site bears an equalized negative pressure, and preventing the tissue fluid stored in the absorbent layer 500 from flowing back to the wound or being sucked upward from the absorbent layer 500. In an embodiment of the present invention, the materials for the second pressure distributing layer 700 and the third pressure distributing layer 800 can be but not limited to natural or synthetic reticulated foam materials, non-woven fabrics or woven fabrics. The materials for the second pressure distributing layer 700 and the third pressure distributing layer 800 can be modified according to different necessity. The materials for the second pressure distributing layer 700 and the third pressure distributing layer 800 can be further treated in order to obtain hydrophilic, hydrophobic or complex characteristics, for example, different regions of the second pressure distributing layer 700 and the third pressure distributing layer 800 are available in hydrophilic or hydrophobic forms. In a preferred embodiment of the present invention, the second pressure distributing layer 700 and the third pressure distributing layer 800 are available in hydrophobic forms to avoid the problem of negative pressure delivery ineffectively caused by tissue fluid blocking.

Accordingly, when using the negative pressure wound dressing of the present invention, the wound is fluid sealed by the barrier layer 100, and the connecting aperture 110 of the barrier layer 100 is fluidly communicated to a negative pressure source. The negative pressure is delivered uniformly to the wound through the first pressure distributing layer 300, the second pressure distributing layer 700, the third pressure distributing layer 800 and the wound contacting layer 600. The tissue fluid of the wound or the wound exudates is sucked into the absorbent layer 500, and the tissue fluid or wound exudates is blocked by the liquid impermeable, gas permeable layer 400 and is stored in the absorbent layer 500. The moisture passing through the liquid impermeable, gas permeable layer 400 is accumulated on the indicator layer 200, which results in the visual indicating element 210 disappearing, changing color, deforming or fading. Therefore, the user can replace the wound dressing timely by the visual indicating element 210 for detecting the humidity, and the problem of suction ineffectively due to excess wound exudates stored in the absorbent layer 500 can be avoided and the wound healing can be promoted thereby.

The advantages of the negative pressure wound dressing of the present invention are as the following:
1. Compared to conventional negative pressure wound therapy, the negative pressure wound dressing of the present invention is more convenient to use and will not affects the movement of patients because no additional liquid collection canister is required.
2. The problem of suction ineffectively due to excess wound exudates stored in the absorbent layer can be avoid because the users can be reminded to replace the wound dressing timely by the visual indicating element.
3. The negative pressure can be distributed evenly and the visual indicating element can be changed evenly. Therefore, the negative wound dressing of the present invention can be used efficiently.

In the manner described above, the users of the negative pressure wound dressing of the present invention can be remind to replace the wound dressing timely to improve the wound healing effectively.

While the invention has been described by way of example(s) and in terms of the preferred embodiment(s), it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A negative pressure wound dressing, comprising:
a barrier layer comprising a connecting aperture for fluidly communicating to a negative pressure source;
an indicating layer stacked on the barrier layer, wherein the indicating layer comprising at least one visual indicating element on the surface adjacent to the barrier layer;
a liquid impermeable, gas permeable layer stacked on the indicating layer;
an absorbent layer stacked on the liquid impermeable, gas permeable layer; and
a wound contacting layer stacked on the absorbent layer.

2. The negative pressure wound dressing according to claim 1, wherein the visual indicating element is a humidity sensing element.

3. The negative pressure wound dressing according to claim 2, wherein the humidity sensing element is a pattern of water based ink.

4. The negative pressure wound dressing according to claim 2, wherein the humidity sensing element is a pattern of humidity sensing agent, an aqueous-triggered color-appearing ink, a hydrochromic ink or soaking discoloration ink.

5. The negative pressure wound dressing according to claim 1, wherein the liquid impermeable, gas permeable layer comprises a plurality of micropores with a diameter in the range of 0.3 μm to 0.6 μm.

6. The negative pressure wound dressing according to claim 1, wherein the liquid impermeable, gas permeable layer is a polymer film selected from at least one of the group consisting of polyethylene, polypropylene, polyvinyl alcohol, polyvinyl acetate, polytetrafluoroethylene, polyvinylidene fluoride, polyvinyl pyrrolidone, polyurethanes, polyamides, polyesters, polyacrylates, polymethacrylate and polyacrylamide or combinations thereof.

7. The negative pressure wound dressing according to claim 1, wherein the absorbent layer is able to hold at least 30 milliliters of fluid.

8. The negative pressure wound dressing according to claim 1, wherein the absorbent layer is the form of fabric, sponge or foam, and made from super absorbent polymers, hydrogel materials or gel materials.

9. The negative pressure wound dressing according to claim 1, wherein the wound contacting layer comprises a plurality of vertical through holes.

10. The negative pressure wound dressing according to claim 1, wherein the material of the wound contacting layer is selected from at least one of the group consisting of polyurethane, polyolefin, vinyl acetate, silicone, fluorosilicone, fluoroelastomers, styrene, butadiene and thermoplastic elastomer or combinations thereof.

11. The negative pressure wound dressing according to claim 1, wherein the barrier layer is an impermeable elastic film.

12. The negative pressure wound dressing according to claim 1, wherein the material of the barrier layer is selected from at least one of the group consisting of natural rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane, ethylene vinyl acetate, co-polyester and silicones or combinations thereof.

13. The negative pressure wound dressing according to claim 1, further comprising a first pressure distributing layer stacked between the indicating layer and the liquid impermeable, gas permeable layer.

14. The negative pressure wound dressing according to claim 13, wherein the material of the first pressure distributing layer is selected from at least one of the group consisting of natural or synthetic reticulated foam materials, non-woven fabrics and woven fabrics.

15. The negative pressure wound dressing according to claim 13, further comprising a second pressure distributing layer stacked between the liquid impermeable, gas permeable layer and the absorbent layer.

16. The negative pressure wound dressing according to claim 15, wherein the material of the second pressure distributing layer is selected from at least one of the group consisting of natural or synthetic reticulated foam materials, non-woven fabrics and woven fabrics.

17. The negative pressure wound dressing according to claim 15, further comprising a third pressure distributing layer stacked between the absorbent layer and the wound contacting layer.

18. The negative pressure wound dressing according to claim 17, wherein the material of the first pressure distributing layer is selected from at least one of the group consisting of natural or synthetic reticulated foam materials, non-woven fabrics and woven fabrics.
